(19) United States
(12) Patent Application Publication
(10) Pub. No.: US 2012/0213871 A1
(43) Pub. Date: Aug. 23, 2012

(54) HERBAL SOLID FORMULATION AND PROCESS FOR PREPARING THE SAME

(75) Inventors: Shankar Kumar MITRA, Bangalore (IN); Uddagiri Venkanna BABU, Bangalore (IN); Ekta SAXENA, Bangalore (IN); Anu Vrat SHARMA, Bangalore (IN)

(73) Assignee: HIMALAYA GLOBAL HOLDINGS LTD, Grand Cayman (KY)

(21) Appl. No.: 13/011,751

(22) Filed: Jan. 21, 2011

Related U.S. Application Data
(63) Continuation-in-part of application No. 13/003,543, filed on Aug. 1, 2011.

(51) Int. Cl. A61K 36/58; A61K 36/67; A61K 36/185

(52) U.S. Cl. 424/734; 424/725; 424/774; 424/779

(57) ABSTRACT

Disclosed is a herbal solid formulation comprising Andrographis paniculata, Terminalia arjuna, Azadirachta indica, Trikatu (Zingiber officinalis, Piper longum, Piper nigrum), Tinospora cordifolia or Ocimum sanctum extracts essentially free of excipients and preservatives and process for preparing the same.

This application is a continuation-in-part of U.S. application Ser. No. 13/003,543 which is a U.S. national stage entry of PCT application number PCT/IB2008/001797 filed Jul. 9, 2008 the entire disclosures of which are expressly incorporated by reference herein.

### FIELD OF THE INVENTION

This invention, in general relates to a herbal solid formulation. In particular, the present invention provides a herbal solid formulation comprising Andrographis paniculata, Terminalia arjuna, Azadirachta indica, Trikatu (Zingiber officinalis, Piper longum, Piper nigrum), Tinospora cordifolia or Ocimum sanctum extracts without using any excipients and preservatives and process for preparing the same.

### BACKGROUND OF THE INVENTION

Herbal supplements have been witnessed tremendous growth and acceptance among the consumers during the last decade due to their safety and efficacy. Unlike allopathic medications, herbal extracts are safe and devoid of any side effects. There is a growing concern among the consumers worldwide using naturally derived products and avoiding synthetic chemicals in their food, personal care products and daily health supplements. Many herbal products those are available in the market as tablets and capsule using synthetic excipients such as binders, lubricants and diluents and preservatives such as Parabens and salts of benzoic acids etc. These excipients and preservatives are reported to have toxic and side effects.

Pharmaceutical dosage forms such as tablets and capsules should have certain properties such as hardness, friability, disintegration time (DT), stability and delivery of the drug to give required therapeutic benefits to the patient. These properties are achieved using the excipients such as binders, lubricants and diluents.

It is therefore very important and challenging task to develop a process of manufacturing herbal tablets using herbal extract and plant powder without using any synthetic excipient and preservative.

### RELATED ART

U.S. Pat. No. 6,207,189 by Mercati et al disclose a process for the production of tablets and capsules of natural substances of vegetable origin wherein dry extracts and micronized powders of one or more medicinal herbs in appropriate proportions are blended and subjected to steam pressure followed by drying, preparation of granules and compression to tablets.

U.S. Pat. No. 6,468,563 by Schmidt et al. discloses a process for producing rapidly disintegrating pharmaceutical formulation containing an extract and lubricant and compressing the blend to form the pharmaceutical formulation.

### SUMMARY OF THE INVENTION

It is a principal object of the invention to provide a herbal solid formulation comprising Andrographis paniculata, Terminalia arjuna, Azadirachta indica, Trikatu (Zingiber officinalis, Piper longum, Piper nigrum) Tinospora cordifolia or Ocimum sanctum extracts essentially free of additives/excipients and preservatives and providing required quantity of active constituents per dose.

It is another object of the invention to provide a herbal solid formulation of herb Andrographis paniculata, Terminalia arjuna, Azadirachta indica, Trikatu (Zingiber officinalis, Piper longum, Piper nigrum), Tinospora cordifolia or Ocimum sanctum extracts having reduced side effects and toxicity.

It is yet another object of the invention to provide a herbal solid formulation of herb Andrographis paniculata, Terminalia arjuna, Azadirachta indica, Trikatu (Zingiber officinalis, Piper longum, Piper nigrum) Tinospora cordifolia or Ocimum sanctum extracts essentially free of excipients/additives and preservatives and having desired friability, disintegration time and hardness.

It is still another object of the invention to provide a method for preparing extract of herb Andrographis paniculata, Terminalia arjuna, Azadirachta indica, Trikatu (Zingiber officinalis, Piper longum, Piper nigrum) Tinospora cordifolia or Ocimum sanctum used to prepare the solid formulation.

The above and other objects of the present invention are attained according to following preferred embodiments of the present invention. However the scope of the invention is not restricted to the particular embodiments discussed herein after.

In accordance with one preferred embodiment of the present invention, there is provided a herbal solid formulation comprising a blend of Super Critical Fluid (CO2) extract, water extract and powder of herb Terminalia arjuna, Azadirachta indica, Trikatu (Zingiber officinalis, Piper longum, Piper nigrum) Tinospora cordifolia or Ocimum sanctum, wherein said blend of extract and said powder of herb is mixed in a ratio of about 1:0.5 to about 1:90.

In accordance with one preferred embodiment of the present invention, there is provided a herbal solid formulation comprises a blend of Super Critical Fluid (CO₂) extract, water extract and powder of herb Terminalia arjuna, Azadirachta indica, Trikatu (Zingiber officinalis, Piper longum, Piper nigrum) Tinospora cordifolia or Ocimum sanctum, wherein said herbal solid formulation is essentially free of additives/excipients.

In accordance with another preferred embodiment of the invention there is provided a process for preparing a herbal solid formulation according to above essentially free of additives/excipients comprises of autoclaving powder of the herb, granulating the autoclaved powder with a water extract of the herb, lubricating the granulated mixture by adding the autoclaved powder of the herb and preparing the solid formulation.

In accordance with yet another preferred embodiment of the invention, the powder of herb is obtained by pulverizing the herb to a powder having mesh size preferably between about 20 to about 100.

In accordance with yet another embodiment of the present invention, the extract of the herb is passed through a mesh having size between about 20 to 80.

In accordance with still another embodiment of the present invention, wherein the granulation of the blend of extracts and powder of the herb is carried out in presence of a solvent, preferably water and grain alcohol or combination thereof.

In accordance with yet another embodiment of the present invention there is provided a process for preparing the extract of the herb by Super Critical Fluid (CO₂) extraction, percolation, hot soxhalation or refluxing followed by filtration and concentration to dryness at optimum temperature.

In accordance with one other embodiment of the present invention, there is provided a process for sterilization of herbal powders by autoclaving the granular mixture, wherein autoclaving prevents microbial growth.

### BRIEF DESCRIPTION OF DRAWINGS

Further objects of the present invention together with additional features contributing thereto and advantages accruing there from will be apparent from the following description of preferred embodiments of the invention which are shown in the accompanying drawing figures, wherein:

FIG. 1 illustrates the LCMS chromatogram of Terminalia Arjuna supercritical fluid CO₂ extract.

FIG. 2 illustrates the LCMS chromatogram of Terminalia Arjuna water extract.

FIG. 3 illustrates LCMS chromatogram of Supercritical fluid CO₂ extract of Azadirachta indica

FIG. 4 illustrates LCMS chromatogram of water extract of Azadirachta indica

FIG. 5 illustrates LCMS chromatogram of water extract of Trikatu FIG. 6 illustrates LCMS chromatogram of Supercritical fluid CO₂ extract of Trikatu FIG. 7 illustrates LC and total ion chromatogram of Guduchi (Tinospora) CO₂ extract.

FIG. 8 illustrates LC and total ion chromatogram of Guduchi (Tinospora) water extract.

FIG. 9 illustrates LC and total ion chromatogram of Tulsi (Ocimum) CO₂ extract.

FIG. 10 illustrates LC and total ion chromatogram of Tulsi (Ocimum) water extract.

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

The present invention provides a herbal solid formulation essentially free of excipients/additives or preservatives, wherein said formulation comprises a blend of Super Critical Fluid (CO₂) extract and water extract of Andrographis paniculata, Terminalia arjuna, Azadirachta indica, Trikatu (Zingiber officinalis, Piper longum, Piper nigrum) Tinospora cordifolia or Ocimum sanctum along with powder of Andrographis paniculata, Terminalia arjuna, Azadirachta indica, Trikatu (Zingiber officinalis, Piper longum, Piper nigrum) Tinospora cordifolia or Ocimum sanctum and a process for preparing the same.

The extracts of the herb is prepared by Super Critical Fluid (CO₂) method. Alternately the same is prepared by employing percolation, hot soxhalation or refluxing method using a solvent, followed by filtration and concentration on a rotatory evaporator on steam bath at optimum temperature and under reduced pressure. The solvent employed includes organic grain alcohol, ethanol or water or combinations thereof, preferably grain alcohol. The extract is dried and passes through a mesh having size preferably between #20 to 80.

The powder of the herb is prepared by pulverizing the root of herb to a powder of different mesh sizes based on the requirement, preferably between about 20 to about 100, more preferably between 20 to 80. The extract and the powder of the herb is mixed in a predetermined ratio preferably between about 1:0.5 to 1:90 for optimum granulation.

The process of preparing the herbal solid formulation involves granulation of the blend of extracts and powder of the herb using a solvent system, followed by passing the granules through a mesh having size preferably between 12 to 24 # and autoclaving the granules. The solvent system employed for granulating the mixture includes grain alcohol, water or combination thereof. Autoclaving helps in microbial control of the solid formulation as it does not contain any preservatives.

The autoclaved granules are further lubricated using the powder of the Andrographis paniculata, Terminalia arjuna, Azadirachta indica, Trikatu (Zingiber officinalis, Piper longum, Piper nigrum) Tinospora cordifolia or Ocimum sanctum and compressed or encapsulated into tablets or capsules.

All extracts, granules and tablets are subjected to standardization by High Performance Thin Layer Chromatography (HPTLC) and High Performance Liquid Chromatography (HPLC) for identification and quantitative estimation of active marker compounds. The extracts were evaluated for toxicity studies in rats and tablets for safety studies in healthy human volunteers.

The solid formulation according to the present invention has desired hardness preferably between 2 to 8 kg/cm², more preferably about 3 to about 4 kg/cm², friability less than about 1% w/w and disintegration time less than about 60 min, preferably between 5 to 60 min, more preferably less than about 30 min. The solid formulation complies with USFDA guidelines.

According to the present invention, the disclosed solid formulation is preferably granules, tablet or capsule.

The following non-limiting examples illustrate specific embodiments of the present invention. They are, not intended to be limiting the scope of present invention in any way.

### Example-1

### Preparation of Andrographis paniculata (Kalmegh) Water Extract

Approximately 100 Kg of shade dried plant material was subjected to extraction with 400 Liters of purified water by percolation method at Room Temperature. The water extractions after 24 hours were filtered through muslin cloth and concentrated to thick paste. After achieving the desired total solid content, the soft extract was spray dried to a free flowing dry extract powder.

### Example-2

### Preparation of Andrographis paniculata (Kalmegh) SCFE (CO₂) Extract

Approximately 25 Kg of The whole plant of Andrographis paniculata was pulverized to fine powder and loaded in to extractor. Super Critical Carbon dioxide liquid was pmped in to the extractor at a pressure of 300 bar and 39° C. temperature for 2-3 hours. Extract was separated into the container at pressure of 40 bar and 20° C. temperature. The CO₂ super critical liquid was recycled from the extraction vessel. The resultant extract was analyzed for active markers of Andrographolides.

### Example-3

### Formula of Granulation

TABLE 1

| S. | | Weight per | Weight per |
|---|---|---|---|
| No. | Name of the material | Tablet (mg) | Batch (Kg) |
| 1 | Andrographis paniculata | 200.00 | 20.00 |
| | (Water extract) (# 40 mesh) | | |
| 2 | Andrographis paniculata | 50.00 | 5.00 |
| | (Super critical fluid (CO2) extract) | | |
| 3 | Andrographis paniculata | 375.00 | 37.50 |
| | (# 40) powder | | |

### Example-4

### Formula of Lubrication of Granules

TABLE 2

| S. | | Weight per | Weight per |
|---|---|---|---|
| No. | Name of the material | Tablet (mg) | Batch (Kg) |
| 1 | Andrographis paniculata Extract | 625.00 | 62.50 |
| | Granules (# 16 mesh) | | |
| 2 | Andrographis paniculata (# 40) | 25.00 | 2.50 |
| | powder | | |
| | Total | 650.00 | 65.00 |

### Example-5

### Manufacturing Procedure of Granulation and Compression of Tablet

1. Dispense Extract, Plant powder as per Batch record.
2. Transfer Andrographis paniculata Leaf powder (#40 mesh) to stainless steel trays and sterilize at 160° C. for 120 minutes.
3. Check the sterilized material for LOD (Loss on Drying), Bulk Density and Microbial growth.
4. Sift 25 Kg of Andrographis paniculata Water extract through #40 mesh and 37.50 Kg and 2.50 Kg of Leaf powder through #40 mesh. Keep the materials packed in double poly bags.
5. Charge 25 Kg of Water extract and 37.50 Kg of Leaf powder in to RMG and mix slowly for 5 minutes. Add 5 Kg of Andrographis paniculata CO₂ extract and 2.5 Kg of Purified water to the mixture over a period of 3 minutes with medium speed.
6. Stop the mixer and scrap off the material from the sides and bottom and continue mixing by operating impeller mixing at high speed with chopper ON for 3 minute.
7. Discharge the material from RMG
8. Mill the wet mass obtained from the above procedure in Multi mill fitted with 8 mm screen.
9. Dry the wet mass obtained through Multi mill at 70° C. to 80° C. for 60 minutes and checks the moisture every 30 minutes to achieve LOD at 2.5 to 3.5%.
10. Sift the dried granules through #16 SS mesh and store in double polylines HDPE containers.
11. Analyze granules for LOD, BD, micro analysis, granules-fine ratio etc.
12. Maintain the temperature at 25° C. and relative humidity NMT 40% of blending and compression area.
13. Transfer the sifted Leaf powder to blending area and blend the leaf powder and granules in to the double cone blender for 3 minutes at 20-25 RPM.
14. Compress the approved granules into tablets with punch size 17×8 mm size with upper and bottom as plain surface.
15. Check appearance, thickness and hardness of tablets every 30 minutes.
16. Check friability and DT for every 1 hour and average weight every 30 minutes.
17. Collect the tablets in double poly lined air tight container.
18. Final tablets have the following specifications:

### Standard Parameters*

1. Theoretical average weight: 650 mg
2. Weight uniformity: 650 mg±5% (617.5 mg to 682.5 mg)
3. Weight of 20 tablets: 13.00 g±3% (12.61 gm to 13.39 gm)
4. Tablet thickness: 4.8 to 5.8 mm
5. Tablet hardness: 4 to 6 Kg/cm⁸²

### 6. Friability: NMT 1.0% W/W

7. Disintegration time: NMT 30 min.
8. Andrographolide: 9 mg per caplet

### Example-6

### Preparation of Terminalia arjuna (Arjuna) Water Extract

Approximately 100 Kg of shade dried plant material bark of Terminalia arjuna was subjected to extraction with 400 Liters of purified water by percolation method at Room Temperature. The water extractions after 24 hours were filtered through muslin cloth and concentrated to thick paste. After achieving the desired total solid content, the soft extract was spray dried to a free flowing dry extract powder. The residual plant materials were named as spent powder.

### Example-7

### Preparation of Terminalia arjuna (Arjuna) SCFE (CO₂) Extract

Approximately 25 Kg of bark of Terminalia arjuna was pulverized to fine powder and loaded in to extractor. Super Critical Carbon dioxide liquid was pumped in to the extractor at a pressure of 300 bar and 39° C. temperature for 2-3 hours. Extract was separated into the container at pressure of 40 bar and 20° C. temperature. The CO₂ super critical liquid was recycled from the extraction vessel.

### Example-8

### Formula of Granulation

### Table-3

| S. | | Weight per | Weight per |
|---|---|---|---|
| No. | Name of the material | Tablet in mg | Batch in Kg |
| 1 | Terminalia arjuna (Water extract) | 240.00 | 24.00 |
| | (# 40 mesh) | | |
| 2 | Terminalia arjuna (Super critical | 10.00 | 1.00 |
| | fluid (CO₂) extract) | | |
| 3 | Terminalia arjuna (spent powder) | 200.00 | 20.00 |
| 4 | Terminalia arjuna leaves (# 40) | 150.00 | 15.00 |
| | powder | | |
| 5 | Purified water | Granulation | Quantity |
| | | fluid | sufficient |

### Example-9

### Formula of Lubrication of Granules

### Table-4

| S. | | Weight per | Weight per |
|---|---|---|---|
| No. | Name of the material | Tablet in mg | Batch in Kg |
| 1 | Terminalia arjuna Extract | 600.00 | 60.00 |
| | Granules (# 16 mesh) | | |
| 2 | Terminalia arjuna spent | 100.00 | 10.00 |
| | powder (# 40) powder | | |
| | Total | 700.00 | 70.00 |

### Example-10

### Dispensing of Raw Material

1. Dispense the raw materials as per Batch Formula

### Dry Heat Sterilization

1. Transfer 30.00 kg of spent powder of Terminalia arjuna bark (left material after water extraction) and 15 kg of leaves of Terminalia arjuna into trays and sterilize the material @ 160° C. for 60 mins.
2. Unload the sterilized materials in to double lined polybags separately and keep in airtight containers.
3. Send the sample for LOD, BD and Microbial Analysis. Table-5.

TABLE 5

| | Parameter | Standard Value | |
|---|---|---|---|
| 1 | LOD | | |
| | spent powder of Terminalia arjuna bark | 2.0-3.0% | w/w |
| | leaves powder of Terminalia arjuna | 2.0-2.30% | w/w |
| 2 | BD | | |
| | spent powder of Terminalia arjuna bark | 0.3-0.5 | g/ml |
| | Leaves powder of Terminalia arjuna | 0.2-0.5 | g/ml |
| 3 | TVAC | NMT 5000 | cfu/gm |
| 4 | Fungal count | NMT 10 | cfu/gm |

### Sifting

1. Sift 24.00 kg of water extract of bark of Terminalia arjuna through #40.
2. Sift 20.00 kg of spent powder of Terminalia arjuna bark and 15.00 kg of Leaves powder of Terminalia arjuna through #60.
3. Sift 6.30 kg of spent powder of Terminalia arjuna bark lubricant through #40 separately.
4. Collect the above-sifted materials in separate duly labeled double lined polybags.
5. Record Quantity Sifted and the sieve integrity before and after sifting.

### Preparation of Granulation Fluid

1. Transfer about 5.0 kg of purified water into a clean Stainless Steel Vessel.
2. Record the observations. Record deviations if any.

### Granulation

1. Charge 15.00 Kg of leaves powder of Terminala arjuna and then add 1.00 kg of super critical (CO₂) extract of Terminalia arjuna for about 5 min.
2. Charge 20.00 kg of spent powder of Terminalia arjuna bark into RMG and blend for about 5 min.
3. Charge 24.00 kg of water extract of bark of Terminalia arjuna into RMG and blend for about 5 min.
4. Slowly add the granulation fluid to the RMG over a period of about 3 min, with medium speed.
5. Stop the mixer and scrape off the mass from the sides and bottom.
6. Continue mixing by operating the impeller at high speed with Chopper ON for about 3 minute.
7. Add additional quantity of purified water, if required.
8. Discharge the mass from the RMG.
9. Record the observations. Record deviations if any.

### Wet Milling

1. Mill the Wet mass in Multi mill fitted with 8 mm screen.

### Tray Drying

1. Dry the Wet mass in tray Drier at about 70° C. to 80° C. for about 60 minutes.
2. Check the Moisture once every 30 minutes. (LOD 4.0 to 6.0% w/w) and record the details.

### Sizing

1. Sift the dried granules using a Sifter fitted with sieve #16.
2. Collect the sifted granules in a clean double poly lined HDPE container.
3. Mill the retains (oversize granules) through a Multi Mill fitted with 1.5 mm screen with ‘Knives Forward’ direction.
4. Pass the milled granules through a Sifter fitted with #16 sieve.
5. Collect the sized granules and add them to the sifted granules.
6. Weigh the granules. Record deviations if any.
7. Analyse samples as per below table-6.
8. If required autoclave the granules at 120° C. for 20 min.

TABLE 6

| | |
|---|---|
| Parameter | Standard values |

| 1 | LOD | 4.0-6.0% | w/w |
|---|---|---|---|
| 2 | BD | 0.40-0.60 | g/ml |
| 3 | Actives | As per finished product spec | |
| 4 | TVAC | NMT 5000 | cfu/gm |
| 5 | Fungal count | NMT 10 | cfu/gm |

### Blending

Maintain the Temperature and Relative Humidity of the area with in the specified limit (Limit Temperature NMT 25° C. and relative humidity NMT 40%)
1. Transfer the sized granules to the blending area.
2. Transfer the sifted spent powder of Terminalia arjuna lubricant to the blending area.
3. Load 10.00 kg of spent powder of Terminalia arjuna lubricant and the sized granules into the Double Cone blender.
4. Blend the ingredients for 3 minutes at 20-25 RPM.
5. Record the details.
6. Unload the blend in a clean Double poly-lined HDPE drums and affix duly filled status labels.
7. Weigh the blend and enter the details.

### Compression

Maintain the Temperature and Relative Humidity of the area with in the specified limit (Limit Temperature NMT 25° C. and relative humidity NMT 40%)
1. Check the Temperature and Relative Humidity of the area and record.
2. Bring the drums containing the blend into the compression area.
3. Adjust the machine.
4. Carry out the initial checks before starting the operation as specified in Table-7.

TABLE 7

| | Description |
|---|---|
| Punch Size | 17 × 8 mm caplet |
| Upper Punch | Plain |
| Lower Punch | Plain |

5. Check for Appearance, Average Weight, Individual weight, Thickness, Hardness, Friability & Disintegration of 6 tablets.
6. Check appearance, thickness & hardness of tablets every 30 min.
7. Check Friability and DT every 1-hour.
8. Check Average weight of 20 tablets every 30 mins graphically.
9. Collect the tablets in a double poly-lined, tightly closed, container. Weigh each container and enter the details.

### Standard Parameters of Tablets

Standard parameters are mentioned in Table-8

TABLE 8

| | | |
|---|---|---|
| Sl. | | |
| No. | Parameters | Standard value |

| 1 | Theoretical average weight | 700 | mg |
|---|---|---|---|
| 2 | Weight uniformity | 700 mg ± 5% | |
| | | (665 mg to 735 mg) | |
| 3 | Weight of 20 tablets | 14.00 g ± 3% | |
| 4 | Tablet thickness | 4.5 to 5.5 | mm |
| 5 | Tablet hardness | 3 to 5 | Kg/cm² |
| 6 | Friability | NMT 1.0% | W/W |
| 7 | Disintegration time | NMT 30 | min. |
| 8 | Arjunolic acid | 0.25 | mg |
| | Tannins | 120 | mg |

1. Collect the Compressed tablets in Double poly lined HDPE drums and record their weights.
2. Affix duly filled status labels to the containers.

### Packing

1. Pack 60 tablets in a HDPE 120 CC container and weigh them for appropriateness of number of tablets.

### Example-11

### Estimation of Arjunolic Acid in Arjuna Dry Extract, Granules

Standard arjunolic acid solution (0.1 mg/ml): Weigh accurately 10 mg of standard arjunolic acid in a 10 ml volumetric flask. Add 7-8 ml of methanol and dissolve. Make the volume up to the mark with methanol. Take 1 ml of this solution in another 10 ml volumetric flask, dilute and make the volume up to the mark with methanol.

Sample preparation (10 mg/ml): Weigh accurately 0.5 g of sample in a 250 ml flat bottomed flask. Add 50 ml of methanol and reflux it on a water bath at 80° C. for 30 minutes. Filter and transfer it into a 50 ml volumetric flask. Make the volume up to the mark with methanol.

HPLC Conditions were as follows

Column: C₁₈ ODS Hypersil (250×4.6) particle size: 5μ, (Make: Thermo)

Mobile phase: Methanol: 0.1% Phosphoric acid (70:30)

Flow rate: 1 ml/minute

Wavelength: 210 nm

Chromatographic procedure: Stabilize the instrument with the above mentioned mobile phase. Inject 20 μl of standard solution and record the chromatogram. Similarly, inject 20 μl of sample solution and record the chromatogram. Calculate the area of standard peak and the corresponding peak in the sample.

Calculation: % w/w Arjunolic acid content can be calculated using the formula.

$$\% w \text{/} w Arjunol ic acid = \frac{Area of sample peak}{Area of standard peak} \times \frac{Concentration of standard ( mg \text{/} ml )}{Concentration of sample ( mg \text{/} ml )} \times \% Purity of standard$$

### Example-12

### LCMSMS Studies

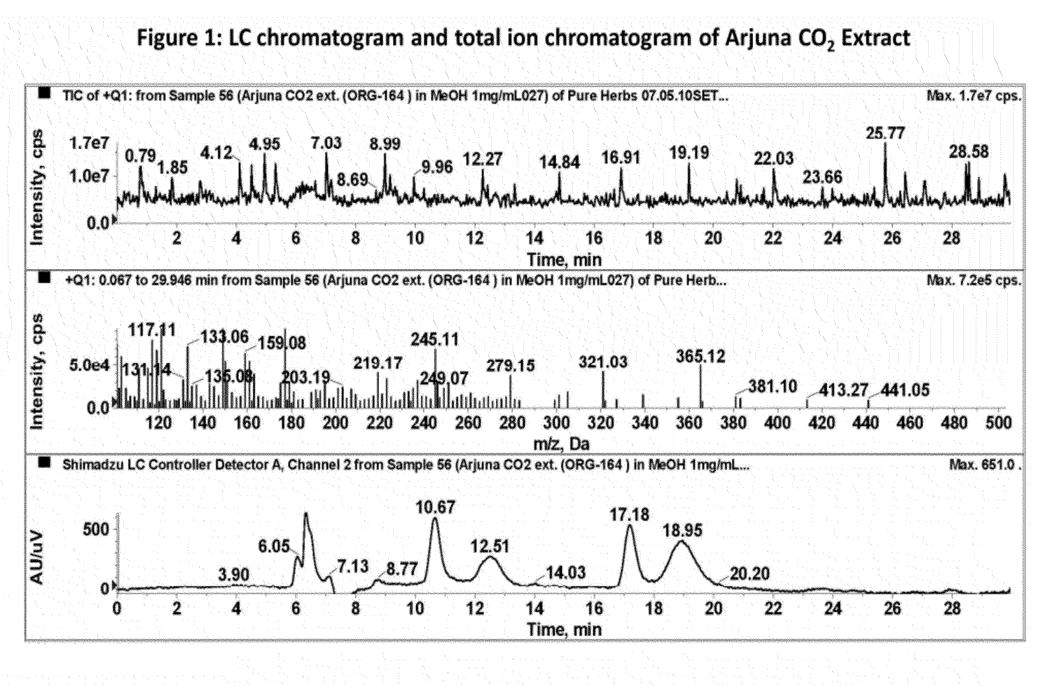

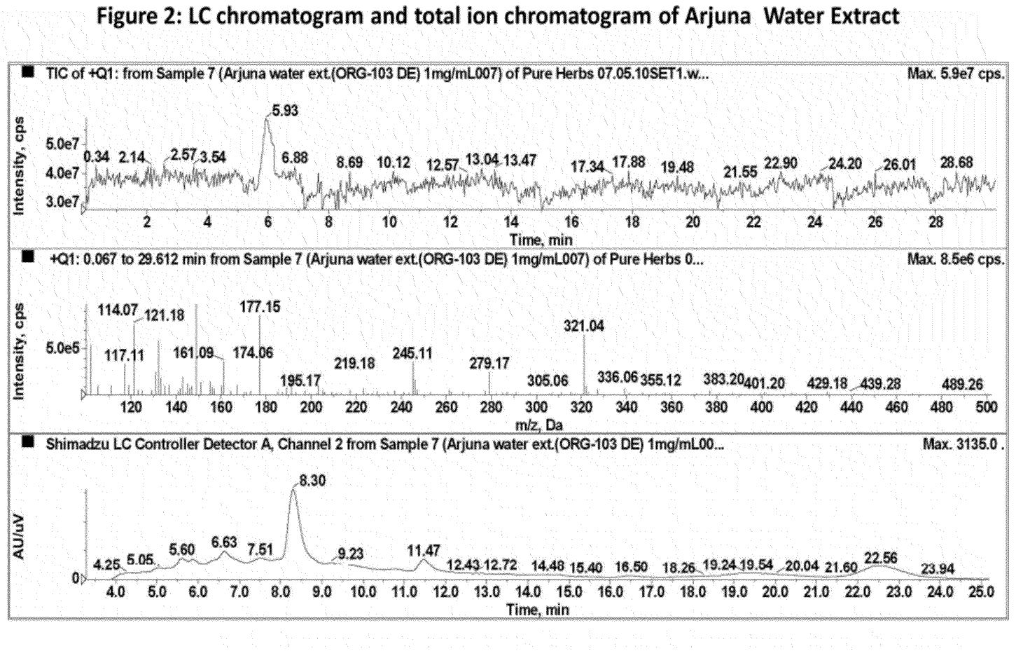

Liquid Chromatography-Mass Spectrometer Analysis of Terminalia arjuna bark supercritical (CO₂) extract and water extract (FIGS. 1 and 2):

LCMSMS analysis were carried out by using an applied biosystem-Sciex API 2000 triple quadrupole mass spectrometer equipped with an ion source turbo spray and ESI interface. The liquid chromatography was a LC-20 AD series binary system equipped with an autosampler. The column used was C18 phenomenex (250×4.6 mm, 5 μm), flow rate 0.5 ml/min of mobile phase methanol: water (0.1% acetic acid), (10:90), wave length 275 nm and run time 25 min. The analytes were ionized by ESI in positive-ion mode (PI mode). Final ionization conditions were heated ionization temperature 435° C. Curtain gas Nitrogen 30 psi, particulate-free and CO₂-free air was used as ion source gas 1 at a flow rate 50 psi and ion source gas 2 at a flow rate 60 psi.

Sample Preparation for Terminalia arjuna Bark Co₂ Extract:

Weigh about 50 mg of sample in a 50 ml volumetric flask, and dissolve by sonication in methanol (HPLC grade). Make the volume up to the mark with methanol filter through 0.2 um syringe filter.

Sample Preparation for Terminalia arjuna Bark Water Extract:

Weigh about 500 mg of finely powdered sample in a 50 ml volumetric flask, and dissolve by sonication in water (HPLC grade). Make the volume up to the mark with water filter through 0.2 um syringe filter.

### Example-13

### Preparation of Azadirachta indica (Neem) Water Extract

Approximately 100 Kg of shade dried plant material leaves of Azadirachta indica was subjected to extraction with 400 Liters of purified water by percolation method at Room Temperature. The water extractions after 24 hours were filtered through muslin cloth and concentrated to thick paste. After achieving the desired total solid content, the soft extract was spray dried to a free flowing dry extract powder.

### Example-14

### Preparation of Azadirachta indica (Neem) SCFE (CO₂) Extract

Approximately 25 Kg of leaves of Azadirachta indica was pulverized to fine powder and loaded in to extractor. Super Critical Carbon dioxide liquid was pumped in to the extractor at a pressure of 300 bar and 39° C. temperature for 2-3 hours. Extract was separated into the container at pressure of 40 bar and 20° C. temperature. The CO₂ super critical liquid was recycled from the extraction vessel.

### Example-15

### Formula of Granulation

### Table-9

| S. | | Weight per | Weight per |
|---|---|---|---|
| No | Name of the material | Tablet in mg | Batch in Kg |
| 1 | Azadirachta indica leaves powder | 200.00 | 20.00 |
| | (Water extract) (# 40) | | |
| 2 | Azadirachta indica leaves (Super | 80.00 | 8.00 |
| | critical fluid (CO2) extract) | | |
| 3 | Azadirachta indica stem (# 100) | 300.00 | 30.00 |
| | powder | | |
| 4 | Purified water | Granulation | Quantity |
| | | fluid | sufficient |

### Example-16

### Formula of Lubrication of Granules

### Table-10

| S. | | Weight per | Weight per |
|---|---|---|---|
| No. | Name of the material | Tablet in mg | Batch in Kg |
| 1 | Azadirachta indica Extract | 580.00 | 58.00 |
| | Granules (# 16 mesh) | | |
| 2 | Azadirachta indica stem powder | 20.00 | 02.00 |
| | (# 100) powder | | |
| | Total | 600.00 | 60.00 |

### Example-17

### Manufacturing Details of Tablets

### Dispensing of Raw Material

1. Dispense the raw materials as per Batch record.

### Dry Heat Sterilization

1. Transfer 30 kg and 2 kg of stem powder of Azadirachta indica into trays and sterilize the material at 160° C. for 60 mins.
2. Unload the materials in to double lined polybags separately and keep in airtight containers.
3. Analyse the sample for LOD, BD and Microbiological Analysis as per Table-11

TABLE 11

| | |
|---|---|
| Parameter | Standard values |

| | | | |
|---|---|---|---|
| 1 | LOD | 2.0-4.0% | w/w |
| 2 | BD | 0.20-0.30 | g/ml |
| 3 | TVAC | NMT 5000 | cfu/gm |
| 4 | Fungal count | NMT 10 | cfu/gm |

### Sifting

1. Sift 20.00 kg of water extract of leaves of Azadirachta indica through #40
2. Sift 08.00 kg of Co₂ extract of leaves Azadirachta indica through #40
3. Sift 30.00 kg of stem powder of Azadirachta indica through #100
4. Sift 2.00 kg of stem powder of Azadirachta indica through #100 separately
5. Collect the above-sifted materials in separate duly labeled double lined polybags.
6. Record the Quantity Sifted and the sieve integrity before and after sifting

### Preparation of Granulation Fluid

1. Transfer about 20.0 kg of Purified water into a clean Stainless Steel Vessel

### Granulation

1. Charge 20.00 Kg of water extract of leaves of Azadirachta indica, 8.0 kg of CO₂ extract of leaves Azadirachta indica and 30.00 kg of stem powder of Azadirachta indica into the RMG, mix for about 5 minute.
2. Slowly add the granulation fluid to the RMG containing the Sifted materials over a period of about 3 minute, with medium speed.
3. Stop the mixer and scrape off the mass from the sides and bottom.
4. Continue mixing by operating the impeller at high speed with Chopper ON for about 3 minute.
5. Add additional quantity of Purified water, if required.
6. Discharge the mass from the RMG.
7. Record the observations. Record deviations if any.

### Wet Milling

1. Mill the Wet Mass in Multi mill fitted with 8 mm screen.

### Tray Drying

1. Dry the Wet mass in tray drier at about 70° C. to 80° C. for about 60 minutes.
2. Check the Moisture once every 30 minutes. (LOD Limit: 3 to 4% w/w) and record the details

### Sizing

1. Sift the dried granules using a Sifter fitted with sieve #16.
2. Collect the sifted granules in a clean double polylined HDPE container.
3. Mill the retains (oversize granules) through a Multi Mill fitted with 1.5 mm screen with ‘Knives Forward’ direction
4. Pass the milled granules through a Sifter fitted with #16 sieve.
5. Collect the sized granules and add them to the sifted granules
6. Weigh the granules.
7. Analyse the sample as per Table-12

TABLE 12

| | |
|---|---|
| Parameter | Standard values |

| 1 | LOD | 2.5-3.5% | w/w |
|---|---|---|---|
| 2 | BD | 0.30-0.40 | g/ml |
| 3 | Granules to fine ration | 70:30 | |
| 4 | TVAC | NMT 5000 | cfu/gm |
| 5 | Fungal count | NMT 10 | cfu/gm |

### Blending

1. Transfer the sized granules into the blending area.
2. Transfer the sifted stem powder of Azadirachta indica lubricant into the blending area.
3. Load 5 kg of stem powder of Azadirachta indica and the sized granules into the Double Cone blender.
4. Blend the ingredients for about 3 minutes at 20-25 RPM.
5. Record the details
6. Unload the blend in a clean Double poly-lined HDPE drums and affix duly filled status labels.
7. Weigh the blend and enter the details.

### Compression

Maintain the Temperature and Relative Humidity of the area with in the specified limit (Limit Temperature NMT 25° C. and relative humidity NMT 40%)
1. Check the Temperature and Relative Humidity of the area and record.
2. Bring the drums containing the blend into the compression area.
3. Adjust the machine as per the parameters.
4. Carry out the initial checks before starting the operation as specified in below Table-13

TABLE 13

| | Description |
|---|---|
| Punch Size | 17 × 8 mm caplet |
| Upper Punch | Plain |
| Lower Punch | Plain |

5. Check for Appearance, Average weight, Individual weight, Thickness, Hardness, Friability & DT of 6 tablets.
6. Check appearance, thickness & hardness of tablets every 30 min.
7. Check Friability and DT every 1-hour.
8. Check Average weight of 20 tablets every 30 mins graphically.
9. Collect the tablets in a double poly-lined, tightly closed, container. Weigh each container and enter the details.

### Standard Parameters of Tablets

Standard parameters are mentioned in Table-14

TABLE 14

| | | |
|---|---|---|
| S. | | |
| No. | Parameters | Standard value |

| 1. | Theoretical average weight | 600 | mg |
|---|---|---|---|
| 2. | Weight uniformity | 600 mg ± 5% | |
| | | (570 mg to 630 mg) | |
| 3. | Weight of 20 tablets | 12.0 g ± 3% | |
| | | (11.64 gm to 12.36 gm) | |
| 4. | Tablet thickness | 4.0 to 5.0 | mm |
| 5. | Tablet hardness | 4 to 6 | Kg/cm² |
| 6. | Friability | NMT 1.0% | W/W |
| 7. | Disintegration time | NMT 30 | min. |

| | | |
|---|---|---|
| 8. | Bitters containing Nimbidin | 18 mg per caplet |

10. Collect the Compressed tablets in Double polylined HDPE drums and record their weights.
11. Affix duly filled status labels to the containers.

### Packing

1. Pack 60 tablets in a HDPE 120 CC container and weigh them for appropriateness of number of tablets.

### Example-18

### LCMSMS Studies

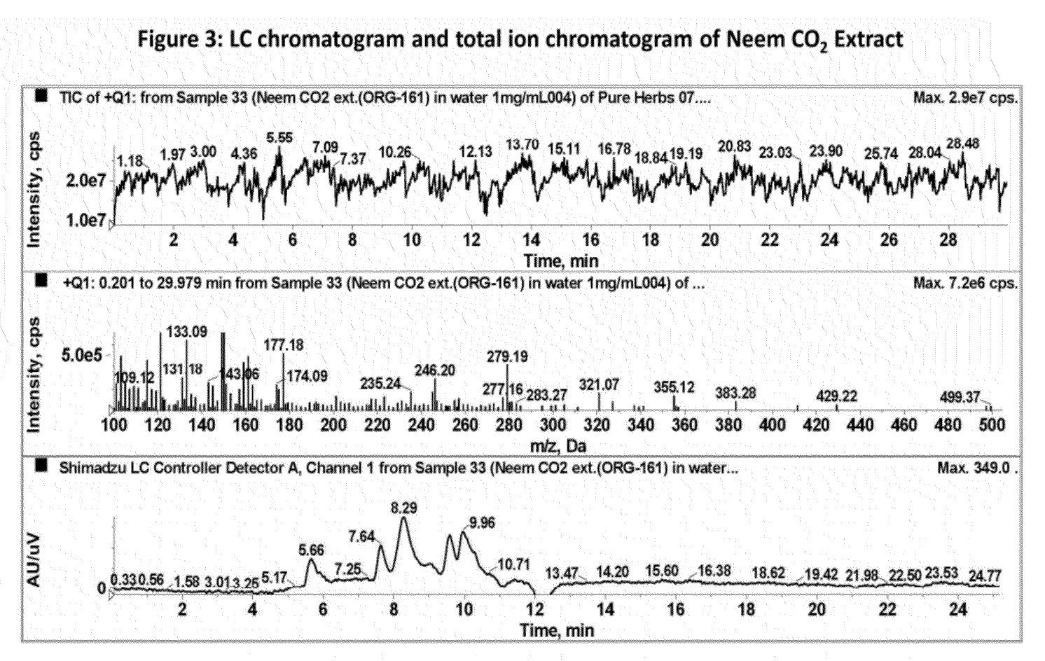

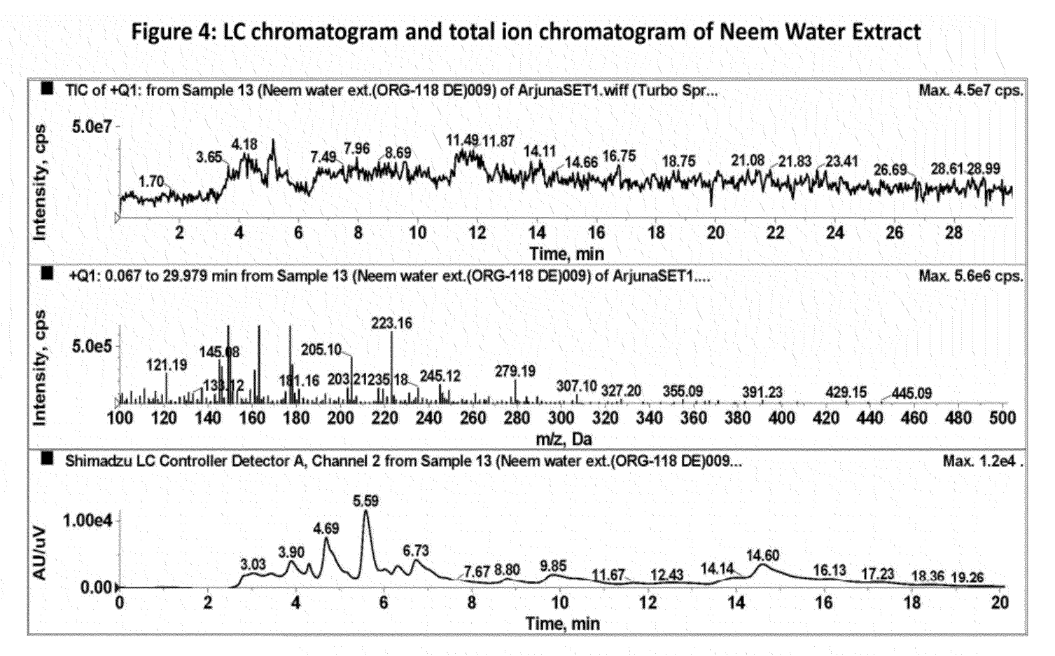

Liquid Chromatography-Mass Spectrometer Analysis of Azadirachta indica leaves, supercritical (CO₂) extract and water extract (FIGS. 3 and 4):

LCMSMS analysis were carried out by using an applied biosystem-Sciex API 2000 triple quadrupole mass spectrometer equipped with an ion source turbo spray and ESI interface. The liquid chromatography was a LC-20 AD series binary system equipped with an autosampler. The column used was C18 phenomenex (250×4.6 mm, 5 μm), flow rate 0.5 ml/min of mobile phase methanol: water (0.1% acetic acid), (10:90), wave length 254 nm and run time 25 min. The analytes were ionized by ESI in positive-ion mode (PI mode). Final ionization conditions were heated ionization temperature 435° C. Curtain gas Nitrogen 30 psi, particulate-free and CO₂-free air was used as ion source gas 1 at a flow rate 50 psi and ion source gas 2 at a flow rate 60 psi.

Sample Preparation for Azadirachta indica Leaves CO₂ Extract:

Weigh about 50 mg of sample in a 50 ml volumetric flask, and dissolve by sonication in methanol (HPLC grade). Make the volume up to the mark with methanol filter through 0.2 um syringe filter.

Sample Preparation for Azadirachta indica Leaves Water Extract:

Weigh about 500 mg of finely powdered sample in a 50 ml volumetric flask, and dissolve by sonication in water (HPLC grade). Make the volume up to the mark with water filter through 0.2 um syringe filter

### Example-19

### Preparation of Trikatu (Zingiber officinale rhizome, Piper longum fruits, Piper nigrum fruits, (1:1:1)) Water Extract

Approximately 100 Kg of shade dried plant material was subjected to extraction with 450 Liters of purified water by percolation method at Room Temperature. The water extractions after 24-48 hours were filtered through muslin cloth and concentrated to thick paste. After achieving the desired total solid content, the soft extract was spray dried to a free flowing dry extract powder. The water extract was also prepared by hot soxhlation method.

### Example-20

### Preparation of Trikatu (Zingiber officinale rhizome, Piper longum fruits, Piper nigrum Fruits, (1:1:1)) SCFE (CO₂) Extract

Approximately 25 Kg of Trikatu material was pulverized to fine powder and loaded in to extractor. Super Critical Carbon dioxide liquid was pumped in to the extractor at a pressure of 325 bar and 32° C. temperature for 2-4 hours. Extract was separated into the container at pressure of 45 bar and 21° C. temperature. The CO₂ super critical liquid was recycled from the extraction vessel.

### Example-21

### Batch Formula for Preparation of Organic Trikatu Granules (Formula-1), Table-15

TABLE 15

| S. | | Weight per | Weight per |
|---|---|---|---|
| No. | Raw Material | Tablet in mg | Batch In kg |
| 1 | Water extract of Trikatu (60 # mesh) | 280.00 | 28.00 |
| 2 | Super critical fluid extract | 20.00 | 2.00 |
| | (SCFE, CO₂ extract) of Trikatu | | |
| 4 | Trikatu plant powder (60 # mesh) | 340.00 | 34.00 |
| 5 | Purified Water | Qs * | Qs * |

### Formula of Lubrication of Trikatu Granules, Table-16

TABLE 16

| | | | | |
|---|---|---|---|---|
| 1 | Trikatu granules | Granules | 640.00 | 64.00 |
| | (# 16 mesh) | | | |
| 2 | Trikatu plant powder | Lubricant | 50.00 | 5.00 |
| | (60 # mesh) | | | |
| | | Total | 690.00 | 69.00 |

### Example-22

### Manufacturing Details of Trikatu Tablets

### Dispensing of Raw Material

1. Dispense the raw materials as per Batch record.

### Dry Heat Sterilization

1. Transfer Trikatu plant powder into trays and sterilize the material at 160° C. for 2 hr.
2. Unload the sterilized material in to double lined poly bags separately and keep in airtight containers.
3. Analyse the sample for LOD, BD and Microbiological Analysis. Table-17

TABLE 17

| | |
|---|---|
| Parameter | Standard values |

| | | | |
|---|---|---|---|
| 1 | LOD | 1.0-4.0% | w/w |
| 2 | BD | 0.25-0.60 | g/ml |
| 3 | TVAC | NMT 5000 | cfu/gm |
| 4 | Fungal count | NMT 10 | cfu/gm |

### Sifting

1. Sift 5.0 kg of water extract powder of Trikatu through #60.
2. Sift 34.0 and 5.0 kg of Trikatu plant powder through #60.
3. Collect the above-sifted materials in separate duly labeled double lined poly bags.

### Preparation of Granulation Fluid

1. Transfer about 10 kg of Purified water into a clean Stainless Steel Vessel.

### Granulation

1. Charge water extract powder of Trikatu, and Trikatu plant powder into the RMG, mix for about 5 minutes.
2. Slowly add super critical fluid extract (CO2) of Trikatu, and granulation fluid to the RMG containing the Sifted extract powder of Trikatu, and Trikatu plant powder over a period of about 3 minute, with medium speed.
3. Stop the mixer and scrape off the mass from the sides and bottom.
4. Continue mixing by operating the impeller at high speed with Chopper ON for about 3 minute.
5. Add additional quantity of granulation fluid, if required.
6. Discharge the mass from the RMG.

### Wet Milling

1. Mill the Wet Mass in Multi mill fitted with 8 mm screen.

### Tray Drying

1. Dry the Wet mass obtained in tray Drier at about 60° C. to 70° C. for about 60 minutes.

### Sizing

1. Sift the dried granules from Sifter fitted with sieve #16.
2. Collect the sifted granules in a clean double poly lined HDPE container.
3. Mill the retains (oversize granules) obtained through a Multi Mill fitted with mm screen with ‘Knives Forward’ direction
4. Pass the milled granules through a Sifter fitted with #16 sieve.
5. Weigh the granules analyse as per Table-18

TABLE 18

| | |
|---|---|
| Parameter | Standard values |

| | | | |
|---|---|---|---|
| 1 | LOD | 2.0-5.0% | w/w |
| 2 | BD | 0.30-0.60 | g/ml |
| 3 | TVAC | NMT 5000 | cfu/gm |
| 4 | Fungal count | NMT 10 | cfu/gm |

### Blending

1. Transfer the sized granules into the blending area.
2. Transfer the sifted trikatu herb powder lubricant into the blending area.
3. Load trikatu herb powder and the sized granules into the Double Cone blender.
4. Blend the ingredients for 6 minutes at 10-11 RPM.
5. Record the details
6. Unload the blend in a clean Double poly-lined HDPE drums and affix duly filled status labels.
7. Weigh the blend and enter the details.

### Compression

1. Check the Temperature and Relative Humidity of the area and record.
2. Bring the drums containing the blend into the compression area.
3. Adjust the machine.
4. Carry out the initial checks before starting the operation as specified in below.

TABLE 19

| | Description |
|---|---|
| Punch Size | 17 × 8 mm caplet |
| Upper Punch | Plain |
| Lower Punch | Plain |

5. Check for Appearance, Average Weight, Individual weight, Thickness, Hardness,
Friability & DT of 6 tablets.
6. Check appearance, thickness & hardness of tablets every 30 min.
7. Check Friability and DT every 1-hour.
8. Check Average weight of 20 tablets every 30 mins graphically.
9. Collect the tablets in a double poly-lined, tightly closed, container. Weigh each container and enter the details.

### Example-23

### Finished Product Specification of Trikatu Per Caplet. Table-20

TABLE 20

| Parameters | Standard Range | |
|---|---|---|
| Theoretical Average weight | 690 | mg |
| Weight uniformity | 690 mg ± 5% | |
| | (655 mg to 725 mg) | |
| Weight of 20 tablets | 13.8 g ± 3% | |
| | (13.39 gm to 14.21 gm) | |
| Tablet thickness | 3.5 to 6.5 | mm |
| Tablet hardness | 2 to 8 | Kg/cm² |
| Friability | NMT 1.0% | W/W |
| Disintegration time | NMT 30 | min |

| | |
|---|---|
| Gingerols | 3 mg per caplet |
| Piperine | 1.09 mg per caplet |

1. Tablets should be packed immediately after compression.
2. The granules can also be filled in capsules.

### Packing

1. Pack 60 Tablets in a HDPE 120 CC Container and Weigh them for Appropriateness of number of tablets.

### Example-24

### Liquid Chromatography

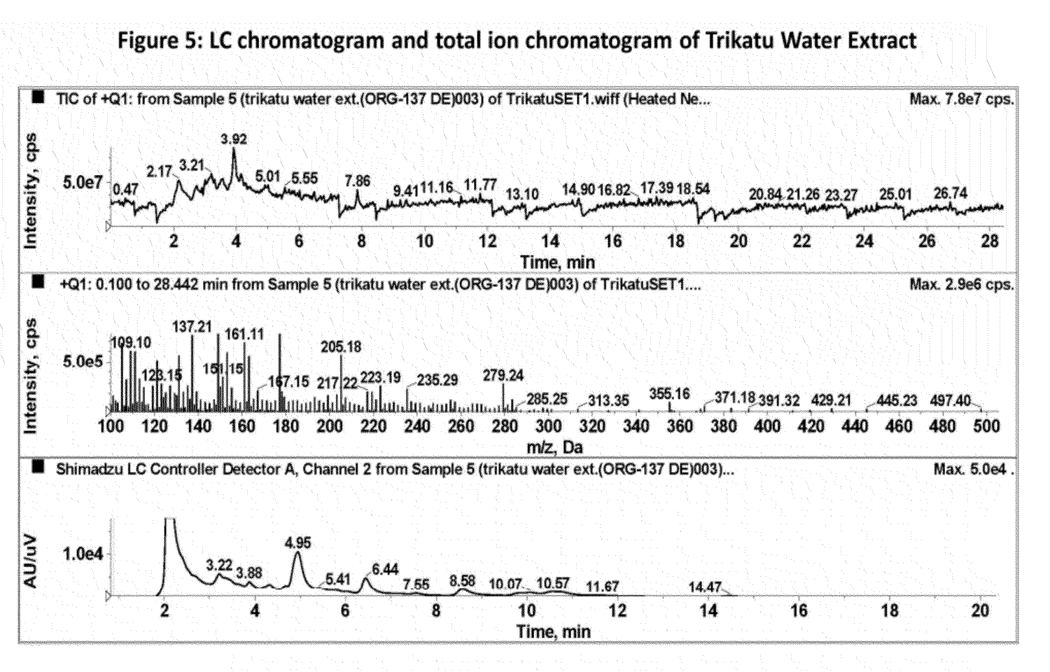

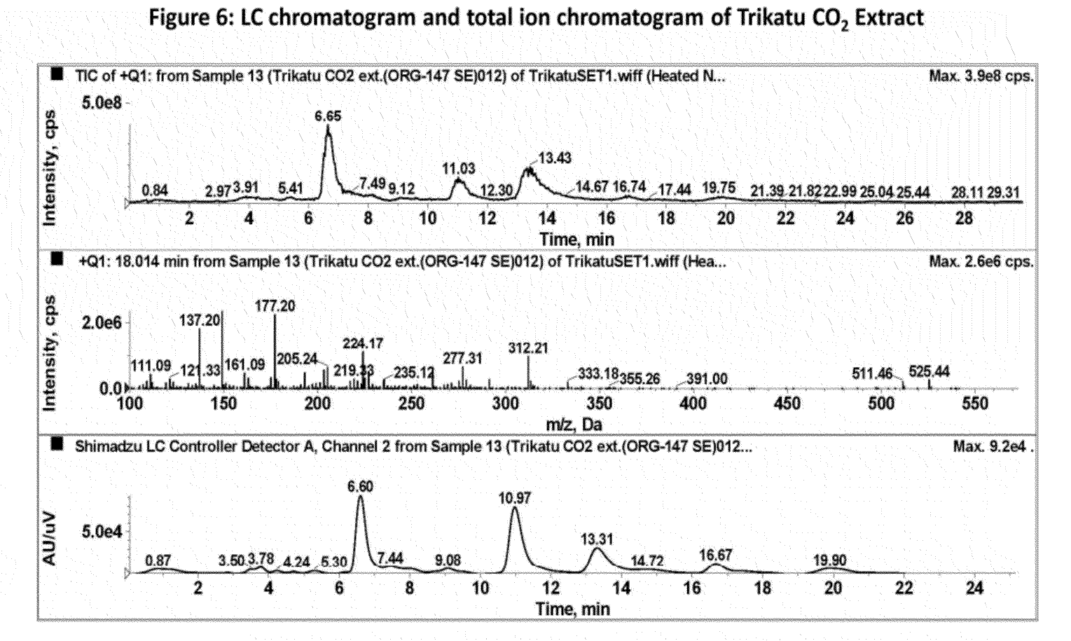

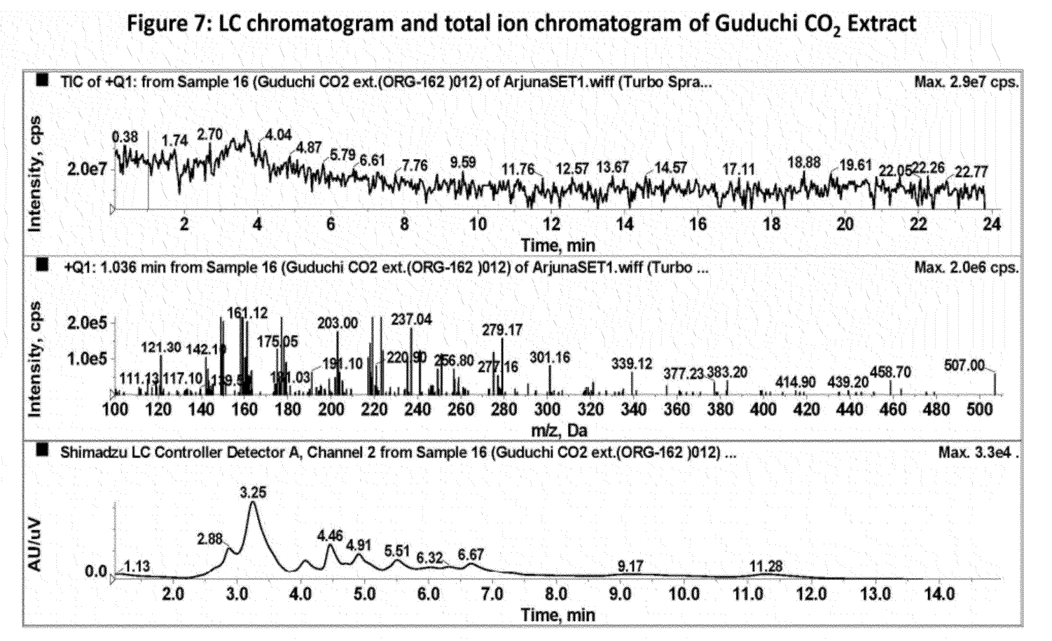

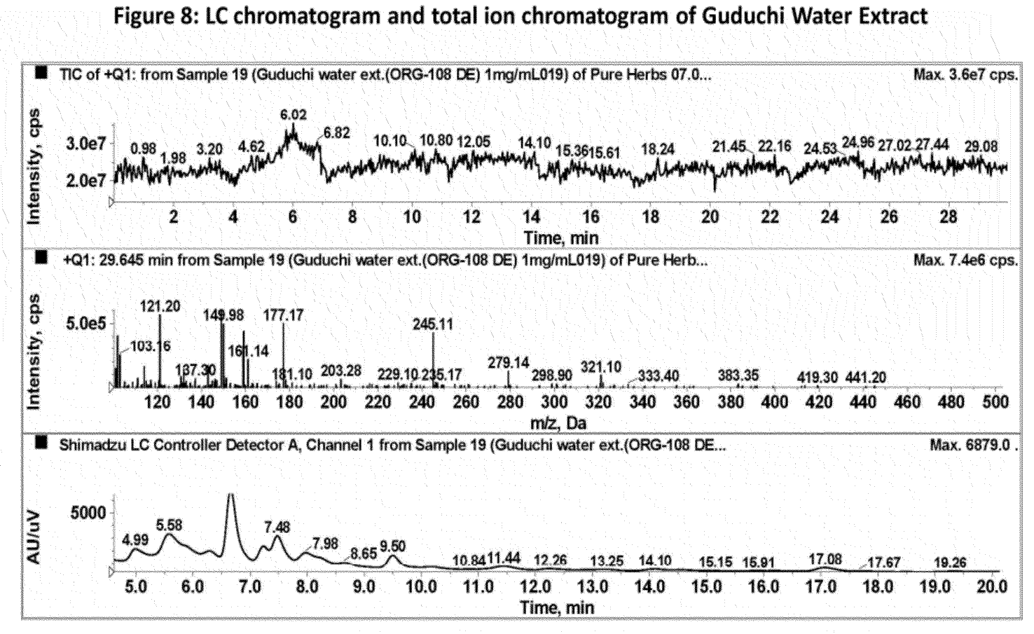

### Mass Spectrometer Analysis of Trikatu Water Extract: (FIG. 5)

LCMSMS analysis was carried out by using an applied biosystem-Sciex API 2000 triple quadrupole mass spectrometer equipped with an atmospheric pressure chemical ionization source and heated nebulizer APCI interface. The liquid chromatography was a LC-20 AD series binary system equipped with an autosampler. The column used was C18 phenomenex (250×4.6 mm, 5 μm), flow rate 1 ml/min of mobile phase water (0.1% acetic acid) (100%), wave length 275 nm and run time 20 min. The analytes were ionized by APCI in positive—ion mode (PI mode). Final ionization conditions were heated nebulizer temperature 450° C., curtain gas Nitrogen 25 psi, particulate-free and CO₂-free air was used as nebulising gas at a flow rate of 70 psi.

### Sample Preparation for Trikatu Water Extract:

Weigh about 500 mg of sample in 50 ml volumetric flask, and dissolve by sonication in water. Make the volume up to the mark with water and filter through 0.2 μm syringe filter. LCMSMS chromatograms are presented in FIG. 5

Liquid Chromatography-Mass Spectrometer Analysis of Trikatu Super Critical Extract (CO₂) extract: (FIG. 6)

LCMSMS analysis were carried out by using an applied biosystem-Sciex API 2000 triple quadrupole mass spectrometer equipped with an atmospheric pressure chemical ionization source and heated nebulizer APCI interface. The liquid chromatography was a LC-20 AD series binary system equipped with an autosampler. The column used was C18 phenomenex (250×4.6 mm, 5 μm), flow rate 1 ml/min of mobile phase methanol: water (0.1% acetic acid); (65:35), wave length 275 nm and run time 20 min. The analytes were ionized by APCI in positive-ion mode (PI mode). Final ionization conditions were heated nebulizer temperature 450° C., curtain gas Nitrogen 25 psi, particulate-free and CO₂-free air was used as nebulising gas at a flow rate of 70 psi.

### Sample Preparation for Trikatu Super Critical Extract (CO₂) Extract:

Weigh about 50 mg of sample in 50 ml volumetric flask, and dissolved by sonication in methanol. Make the volume up to the mark with methanol and filter through 0.2 μm syringe filter. LCMSMS chromatogram are presented in FIG. 6

### Example-25

### Preparation of Tinospora cordifolia (Guduchi) Water Extract

Approximately 100 Kg of shade dried plant material stem was subjected to extraction with 400 Liters of purified water by percolation method at Room Temperature. The water extractions after 24 hours were filtered through muslin cloth and concentrated to thick paste. After achieving the desired total solid content, the soft extract was spray dried to a free flowing dry extract powder.

### Example-26

### Preparation of Tinospora cordifolia (Guduchi) SCFE (CO₂) Extract

Approximately 25 Kg of the stem of Tinospora cordifolia was pulverized to fine powder and loaded in to extractor. Super Critical Carbon dioxide liquid was pumped into the extractor at a pressure of 300 bar and 39° C. temperature for 2-3 hours. Extract was separated into the container at pressure of 40 bar and 20° C. temperature. The CO₂ super critical liquid was recycled from the extraction vessel.

### Example-27

### Formula of Granulation

### Table-21

| S. | | Weight per | Weight per |
|---|---|---|---|
| No. | Name of the material | Tablet in mg | Batch in Kg |
| 1 | Tinospora cordifolia stem | 200.00 | 20.00 |
| | (Water extract) (# 40 mesh) | | |
| 2 | Tinospora cordifolia stem | 50.00 | 5.00 |
| | (Super critical fluid (CO₂) extract) | | |
| 3 | Tinospora cordifolia stem | 400.00 | 40.00 |
| | (# 40 mesh) powder | | |
| 4 | Purified water | Granulation | Quantity |
| | | fluid | sufficient |

### Example-28

### Formula of Lubrication of Granules

### Table-22

| S. | | Weight per | Weight per |
|---|---|---|---|
| No. | Name of the material | Tablet in mg | Batch in Kg |
| 1 | Tinospora cordifolia Extract | 650.00 | 65.00 |
| | Granules (# 16 mesh) | | |
| 2 | Tinospora cordifolia stm | 50.00 | 5.00 |
| | powder (# 80) powder | | |
| | Total | 700.00 | 70.00 |

### Example-29

### Dispensing of Raw Material

1. Dispense the raw materials as per Batch Formula.

### Dry Heat Sterilization

1. Transfer 45.0 kg of stem powder of Tinospora cordifolia in trays and sterilize the materials at 160° C. for 2 hour.
2. Unload the sterilized materials in to double-lined poly bags separately and keep in air tight containers.
3. Analyse the sample for LOD, BD and microbial analysis. Table-23

TABLE 23

| | |
|---|---|
| Parameter | Standard values |

| 1 | LOD | 1.0-4.0% | |
|---|---|---|---|
| 2 | BD | 0.25-0.60 | g/ml |
| 3 | TVAC | NMT 5000 | cfu/gm |
| 4 | Fungal Count | NMT 10 | cfu/gm |

### Sifting

1. Sift 20.0 kg of water extract of stem of Tinospora cordifolia through #40.
2. Sift 5.0 kg of water extract of stem of Tinospora cordifolia through #40.
3. Sift 40.0 kg of stem plant powder through #80.
4. Sift 5.00 kg of stem plant powder lubricant through #80 separately.
5. Collect the above-sifted materials in separate duly labeled double lined polybags.
6. Record Quantity Sifted and the sieve integrity before and after sifting.

### Preparation of Granulation Fluid

1. Transfer about 20.0 kg of Purified water into a clean Stainless Steel Vessel

### Granulation

1. Charge 20.00 Kg of water extract of stem of Tinospora cordifolia, 5.0 kg of super critical extract (CO₂) of stem of Tinospora cordifolia, 40.0 kg of stem plant powder into the RMG, mix for 5 minute.
2. Slowly add the granulation fluid to the RMG containing the Sifted water extract of stem of Tinospora cordifolia, stem plant powder over a period of about 3 minute, with medium speed.
3. Stop the mixer and scrape off the mass from the sides and bottom.
4. Continue mixing by operating the impeller at high speed with Chopper ON for about 3 minute.
5. Add additional quantity of purified water, if required.
6. Discharge the mass from the RMG.

### Wet Milling

2 Mill the Wet Mass obtained in Multi mill fitted with 8 mm screen.

### Tray Drying

1. Dry the Wet mass obtained in tray Drier at about 70° C. to 80° C. for about 60 minutes.
2. Check the Moisture once every 30 minutes. (LOD Limit: 2.0 to 5.0% w/w) and record the details

### Sizing

1. Sift the dried granules using a Sifter fitted with sieve #16.
2. Collect the sifted granules in a clean double poly-lined HDPE container.
3. Mill the retains (oversize granules) obtained through a Multi Mill fitted with 1.5 mm screen with ‘Knives Forward’ direction.
4. Pass the milled granules obtained through a Sifter fitted with #16 sieve.
5. Collect the sized granules obtained.
6. Weigh the granules.
7. Analyse samples as per below Table-24

TABLE 24

| | |
|---|---|
| Parameter | Standard value |

| 1 | LOD | 2.0-5.0% | |
|---|---|---|---|
| 2 | BD | 0.30-0.60 | g/ml |
| 3 | Granules:Fine ratio | 20:80-80:20 | |
| 4 | TVAC | NMT 5000 | cfu/gm |
| 5 | Fungal count | NMT 10 | cfu/gm |

### Blending

1. Transfer the sized granules to the blending area.
2. Transfer the sifted stem plant powder of Tinospora cordifolia lubricant to the blending area.
3. Load 5.0 kg of stem plant powder of Tinospora cordifolia and the sized granules into the double cone blender.
4. Blend the ingredients for about 6 minutes at 10-11 RPM.
5. Unload the blend in a clean Double poly-lined HDPE drums and affix duly filled status labels.
6. Weigh the blend and enter the details.

### Compression

Maintain the temperature and relative humidity of the area with in the specified limit (Limit temperature NMT 25° C. and relative humidity NMT 40%)
1. Check the Temperature and Relative humidity of the area and record.
2. Bring the drums containing the blend into the compression area.
3. Adjust the machine.
4. Carry the initial checks before starting the operation as specified in below table-25

TABLE 25

| | Description |
|---|---|
| Punch size | 17 × 8 mm caplet |
| Upper punch | Plain |
| Lower punch | Plain |

5. Check for Appearance, Average Weight, Individual Weight, Thickness, Hardness, and Friability & DT of 6 tablets.
6. Check appearance, thickness & hardness of tablets for every 30 min.
7. Check friability and DT every 1-hour
8. Check for the Average weight of 20 tablets every 30 mins graphically
9. Collect the tablets in a double poly-lined, tightly closed container. Weigh each container and enter the details.

### Standard Parameters for Tablets

TABLE 26

| | | |
|---|---|---|
| Sl. | | |
| No. | Parameters | Standard value |

| 1 | Theoretical average weight | 700 | mg |
|---|---|---|---|
| 2 | Weight uniformity | 700 mg ± 5% | |
| | | (665 mg to 735 mg) | |
| 3 | Weight of 20 tablets | 14.00 g ± 3% | |
| | | (13.58 gm to 14.42 gm) | |
| 4 | Tablet thickness | 3.5 to 6.5 | mm |
| 5 | Tablet hardness | 2 to 8 | Kg/cm² |
| 6 | Friability | NMT 1.0% | W/W |
| 7 | Disintegration time | NMT 30 | min. |

| | | |
|---|---|---|
| 8 | Total Bitters containing | 24.6 mg per caplet |
| | Tinosporaside | |

Tablets to be packed immediately after compression.

### Packing

2. Pack 60 tablets in a HDPE 120 CC container and weigh them for appropriateness of number of tablets.

### Example-30

### LCMSMS Studies

Liquid Chromatography-Mass Spectrometer Analysis of Tinospora cordifolia stem supercritical (CO₂) extract and water extract (FIGS. 7 & 8):

LCMSMS analysis were carried out by using an applied biosystem-Sciex API 2000 triple quadrupole mass spectrometer equipped with an ion source turbo spray and ESI interface. The liquid chromatography was a LC-20 AD series binary system equipped with an autosampler. The column used was C18 phenomenex (250×4.6 mm, 5 μm), flow rate 0.5 ml/min of mobile phase methanol: water (0.1% acetic acid), (10:90), wave length 254 nm and run time 25 min. The analytes were ionized by ESI in positive-ion mode (PI mode). Final ionization conditions were heated ionization temperature 435° C. Curtain gas Nitrogen 30 psi, particulate-free and CO₂-free air was used as ion source gas 1 at a flow rate 50 psi and ion source gas 2 at a flow rate 60 psi.

Sample preparation for Tinospora cordifolia stem CO₂ extract:

Weigh about 50 mg of sample in a 50 ml volumetric flask, and dissolve by sonication in methanol (HPLC grade). Make the volume up to the mark with methanol filter through 0.2 um syringe filter.

Sample preparation for Tinospora cordifolia stem water extract:

Weigh about 500 mg of finely powdered sample in a 50 ml volumetric flask, and dissolve by sonication in water (HPLC grade). Make the volume up to the mark with water filter through 0.2 um syringe filter.

### Example-31

### Preparation of Ocimum sanctum Water Extract

Approximately 100 Kg of shade dried leaves of (Ocimum sanctum) was subjected to extraction with 400 Liters of purified water by percolation method at Room Temperature. The water extractions after 24 hours were filtered through muslin cloth and concentrated to thick paste. After achieving the desired total solid content, the soft extract was spray dried to a free flowing dry extract powder.

### Example-32

### Preparation of Ocimum sanctum SCFE (CO₂) Extract

Approximately 25 Kg of leaves of Ocimum sanctum was pulverized to fine powder and loaded in to extractor. Super Critical Carbon dioxide liquid was pumped in to the extractor at a pressure of 350 bar and 40° C. temperature for 2-4 hours. Extract was separated into the container at pressure of 40 bar and 20° C. temperature. The CO₂ super critical liquid was recycled from the extraction vessel.

### Example-33

### Formula of Granulation for Tulsi Capsules

### Table-27

| S. | | Weight per | Weight per |
|---|---|---|---|
| No | Name of the material | Tablet in mg | Batch in Kg |
| 1 | Ocimum sanctum leaves powder | 160.00 | 16.00 |
| | (Water extract) (# 40) | | |
| 2 | Ocimum sanctum leaves | 60.00 | 6.00 |
| | (Super critical fluid (CO₂) extract) | | |
| 3 | Ocimum sanctum leaves (# 80 mesh) | 500.00 | 50.00 |
| | powder | | |
| 4 | Purified water | Granulation | Quantity |
| | | fluid | sufficient |

### Example-34

### Formula of Lubrication of Granules for Tulsi Capsules

### Table-28

| S. | | Weight per | Weight per |
|---|---|---|---|
| No. | Name of the material | Tablet in mg | Batch in Kg |
| 1 | Ocimum sactum Extract Granules | 720.00 | 72.00 |
| | (# 16 mesh) | | |
| 2 | E VEG CAPSULES CT/CT “00” | | 100000 |
| | WI/OUT LOGO, empty veg capsule | | |
| | Fill weight | 720.00 | 72.00 |

### Example-35

### Formula of Granulation for Tulsi Tablets

### Table-29

| S. | | Weight per | Weight per |
|---|---|---|---|
| No | Name of the Material | Tablet in mg | Batch In kg |
| 1 | Ocimum sanctum leaves powder | 220.00 | 22.00 |
| | (Water extract) (# 40) Dry extract | | |
| 2 | Ocimum sanctum leaves (Super | 30.00 | 3.00 |
| | critical fluid (CO2) extract) | | |
| 3 | Ocimum sanctum leaves (# 80 | 400.00 | 40.00 |
| | mesh) powder | | |

| | | |
|---|---|---|
| 4 | Granulation fluid | Quantity sufficient |

### Example-36

### Formula of Lubrication of Granules for Tulsi Tablets

### Table-30

| | | | | |
|---|---|---|---|---|
| 1 | Ocimum sanctum leaves granules | Granules | 650.00 | 65.00 |
| | (# 16 mesh) | | | |
| 2 | Ocimum sanctum leaves (# 80 | Lubricant | 50.00 | 5.00 |
| | mesh) powder | | | |
| | | Total | 700.00 | 70.00 |

### Example-37

### Manufacturing details of Tulsi Capsules

### Dispensing of Raw Material

1. Dispense the raw materials as per Batch Formula.

### Dry Heat Sterilization

1. Transfer 50.0 kg of leaves powder of Ocimum sanctum in to trays and sterilize the material at 160° C. for 60 mins
2. Unload the sterilized materials in to double lined polybags separately and keep in airtight containers.
3. Analyze the sample for LOD, BD and Microbiological Analysis Table-31

TABLE 31

| | | |
|---|---|---|
| S. | | |
| No | Parameter | Standard values |

| 1 | LOD | 2.5-3.5% | |
|---|---|---|---|
| 2 | BD | 0.30-0.40 | g/ml |
| 3 | Microbes | NMT 10000 | cfu/gm |
| 4 | Fungal count | NMT 100 | cfu/gm |

### Sifting

1. Sift water extract of leaves powder of Ocimum sanctum through #40 and leaves powder of Ocimum sanctum through #80, weigh as per the required quantity and kept separately in duly-labeled double lined poly bag.
2. Record Quantity Sifted and the sieve integrity before and after sifting.

### Preparation of Granulation Fluid

1. Transfer about 20.0 kg of Purified water into a clean Stainless Steel Vessel
2. Record the observations. Record deviations if any.

### Granulation

1. Charge 50.00 Kg of leaves powder of Ocimum sanctum, add slowly 6.0 kg of super critical extract (CO₂) of leaves of Ocimum sanctum into the RMG mix for 5 minutes.
2. Add 16.00 kg of water extract of leaves of Ocimum sanctum to the above blend. Mix it for 5 minutes.
3. Add granulation fluid to RMG and granulate. If required add additional quantity of purified water, mix over a period of about 3 minutes, with medium speed.
4. Stop the mixer and scrape off the mass from the sides and bottom.
5. Continue mixing by operating the impeller at high speed with Chopper ON for 1 minute.
6. Add additional quantity of purified water, if required.
7. Discharge the mass from the RMG.
8. Record the observations. Record deviations if any.

### Wet Milling

1. Mill the Wet Mass in Multi mill fitted with 8 mm screen.

### Drying

1. Dry the Wet in FBD/Tray drier at about 55-60° C. for about 60-90 minutes.
2. Check the Moisture once every 30 minutes. (LOD Limit: 2-4%) and record the details.

### Sizing

1. Sift the dried granules using a Sifter fitted with sieve #18.
2. Collect the sifted granules in a clean double poly-lined HDPE container.
3. Mill the retains (oversize granules) through a Multi Mill fitted with 1.5 mm screen with ‘Knives Forward’ direction.
4. Pass the milled granules through a Sifter fitted with #16 sieve.
5. Analyze the sample.
6. In-process parameters are in Table-32

TABLE 32

| | |
|---|---|
| Parameter | Standard values |

| 1 | LOD | 1-2% | |
|---|---|---|---|
| 2 | BD | 0.60-0.70 | g/ml |
| 3 | Granules to fine ratio | 60:40 to 70:30 | |
| 4 | Actives | As per Finished | |
| | | Product spec | |
| 5 | Microbes | NMT 10000 | cfu/gm |
| 6 | Fungal count | NMT 100 | cfu/gm |

### Capsule Filling

Note: Maintain the Temperature and Relative Humidity of the area within the specified limit (Temperature 25° C.±2° C. and Relative humidity 40%±2%)
   1. Check the Temperature and Relative Humidity of the area and record.
   2. Bring the drums containing the granules into the capsule filling area.
   3. Adjust the machine.
   4. Check for Appearance, Average Weight, Individual weight, average locking length of 10 capsules& DT of 6 capsules.
   5. Check DT every 1-hour.
   6. Check Average weight of 20 capsules every 30 min graphically.
   7. Collect the capsules in a double poly-lined, tightly closed, container.
   8. Weigh each container and enter the details.
   9. In process specification for capsules Table-33

TABLE 33

| Parameter | Standard limit | |
|---|---|---|
| Description | Clear Transparent size 00 capsules | |
| | filled with brown colored granules | |
| Weight of empty capsules | 115-120 | mg |
| Fill weight | 720 | mg |
| Average weight | 840 ± 5% | |
| Weight 20 capsules | 16.8 gm ± 3% | |
| Average locking length | 23.3 ± 0.4 mm | |
| of 10 capsules | | |
| Disintegration | NMT 15 | min |

8. Collect the filled capsules in double poly-lined HDPE drums and record their weights.
9. Affix duly filled status labels to the containers.

### Packing

1. Pack 60 capsules in a HDPE 120 CC container and weigh them for appropriateness of number of capsules

### Example-38

### Manufacturing Details of Tulsi Tablets

### Dry Heat Sterilization

1. Transfer the leaves plant powder of Ocimum sanctum into trays and sterilize the material at 160° C. for 60 minutes.
2. Unload the autoclaved materials in to double lined polybags separately and keep in airtight containers.
3. Analyse sample for LOD, BD and Microbiological Analysis as per Table-34

TABLE 34

| | |
|---|---|
| Parameter | Standard values |

| 1 | LOD | 2.0-3.0% | |
|---|---|---|---|
| 2 | BD | 0.35-0.55 | g/ml |
| 3 | TVAC | NMT 5000 | cfu |
| 4 | Fungal count | NMT 10 | cfu |

### Sifting

1. Sift water extract of leaves of Ocimum sanctum through #40 mesh.
2. Sift leaves plant powder of Ocimum sanctum through #80 mesh.
3. Collect the above-sifted materials in separate duly labeled double lined polybags.

### Preparation of Granulation Fluid

1. Transfer purified water into a clean Stainless Steel Vessel

### Granulation

1. Charge of water extract of leaves of Ocimum sanctum and of leaves powder of Ocimum sanctum into the RMG, mix for 5 minutes.
2. Slowly add super critical extract (CO₂ extract) of leaves of Ocimum sanctum, and mix for about 3 minutes.
3. Add granulation solution to the above blend and mix for about 3 minutes, with medium speed.
4. Stop the mixer and scrape off the mass from the sides and bottom.
5. Continue mixing by operating the impeller at high speed with Chopper ON, if required.
6. Add additional quantity of Purified water, if required.
7. Discharge the mass from the RMG.

### Wet Milling

1. Mill the Wet Mass obtained in Multi mill fitted with 8 mm screen.

### Tray Drying

1. Dry the Wet mass in tray Drier at about 70° C. to 80° C. for about 60 minutes.

### Sizing

1. Sift the dried granules using a Sifter fitted with sieve #16.
2. Collect the sifted granules in a clean double poly-lined HDPE container.
3. Mill the retains (oversize granules) through a Multi Mill fitted with 1.5 mm screen with ‘Knives Forward’ direction.
4. Pass the milled granules obtained through a Sifter fitted with #16 sieve.
5. Collect the sized granules obtained and add them to the sifted granules.

### Blending

Granules were Analysed as Per Table-35

TABLE 35

| | |
|---|---|
| Parameter | Standard values |

| | | | |
|---|---|---|---|
| 1 | LOD | 3.0-4.0% | w/w |
| 2 | BD | 0.35-0.55 | g/ml |
| 3 | TVAC | NMT 5000 | cfu |
| 4 | Fungal count | NMT 100 | cfu |

Maintain the Temperature and Relative Humidity of the area within the specified limit (Limit Temperature NMT 25° C. and relative humidity NMT 40%)
1. Transfer the sized granules into the blending area.
2. Transfer the sifted leaves powder of Ocimum sanctum lubricant into the blending area.
3. Load 5.0 kg of leaves powder of Ocimum sanctum and the sized granules into the Double Cone blender.
4. Blend the ingredients for 5 minutes at 10-15 RPM.
5. Unload the blend in a clean Double poly-lined HDPE drums and affix duly filled status labels.
6. Weigh the blend and enter the details.

### Compression

Maintain the Temperature and Relative Humidity of the area with in the specified limit (Limit Temperature NMT 25° C. and relative humidity NMT 40%)
5. Check the Temperature and Relative Humidity of the area and record.
6. Bring the drums containing the blend into the compression area.
7. Adjust the machine as per the parameters.
8. Carry out the initial checks before starting the operation as specified in below Table-36

TABLE 36

| | Description |
|---|---|
| Punch Size | 17 × 8 mm caplet |
| Upper Punch | Plain |
| Lower Punch | Plain |

10. Check for Appearance, Average weight, Individual weight, Thickness, Hardness, Friability & DT of 6 tablets.
11. Check appearance, thickness & hardness of tablets every 30 min.
12. Check Friability and DT every 1-hour.
13. Check Average weight of 20 tablets every 30 mins graphically.
14. Collect the tablets in a double poly-lined, tightly closed, container. Weigh each container and enter the details.

### Standard Parameters of Tablets

Standard parameters are mentioned in Table-37

TABLE 37

| | | |
|---|---|---|
| S. | | |
| No. | Parameters | Standard value |

| 1 | Theoretical average weight | 700 | mg |
|---|---|---|---|
| 2 | Weight uniformity | 700 mg ± 5% | |
| | | (705 mg to 695 mg) | |
| 3 | Tablet thickness | 4.0 to 5.0 | mm |
| 4 | Tablet hardness | 4 to 6 | Kg/cm² |
| 5 | Friability | NMT 1.0% | W/W |
| 6 | Disintegration time | NMT 30 | min. |

| | | |
|---|---|---|
| 7 | Total Oleonolic acids | 3.5 mg per caplet |

12. Collect the Compressed tablets in Double poly-lined HDPE drums and record their weights.
13. Affix duly filled status labels to the containers.

### Packing

14. Pack 60 tablets in a HDPE 120 CC container and weigh them for appropriateness of number of tablets.

### Example-39

### Liquid Chromatography

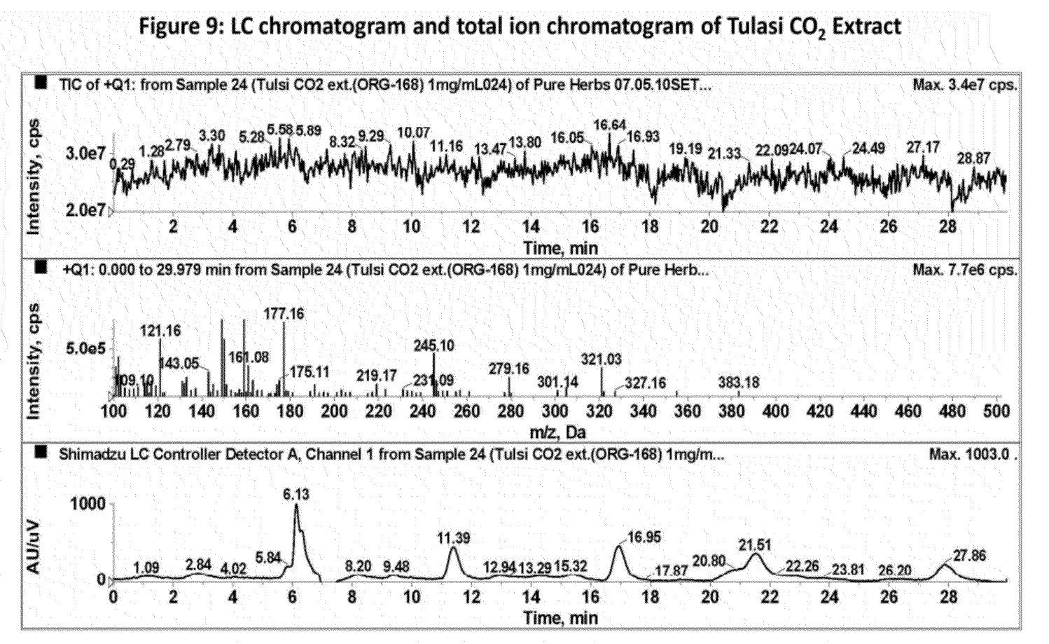

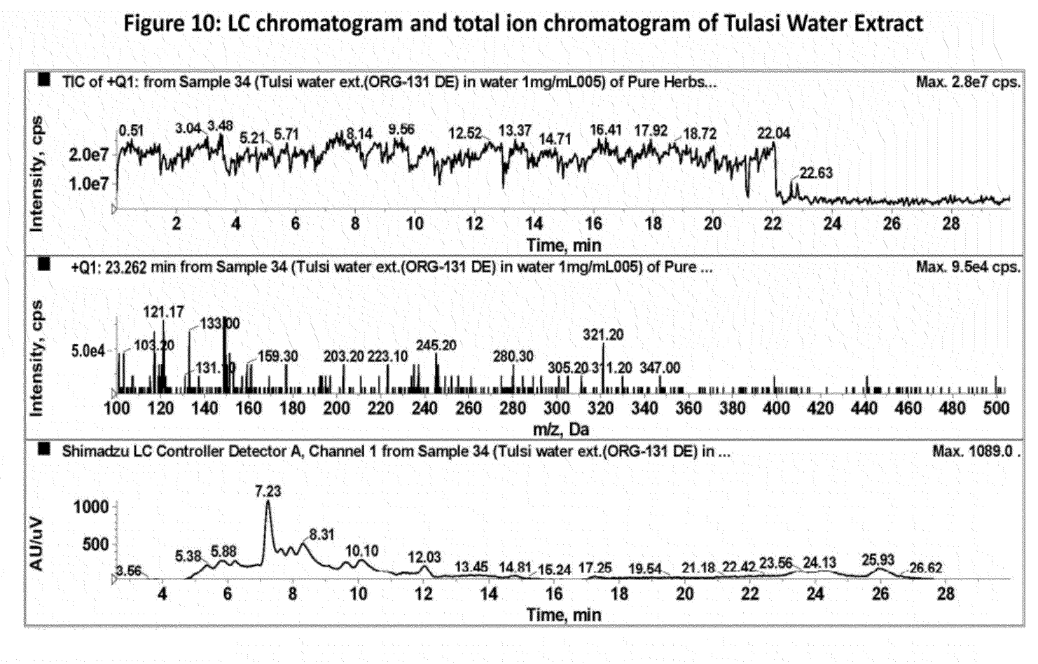

### Mass Spectrometer Analysis of Ocimum sanctum Leaves, Supercritical (CO₂) Extract and Water Extract (FIGS. 9 and 10):

LCMSMS analysis were carried out by using an applied biosystem-Sciex API 2000 triple quadrupole mass spectrometer equipped with an ion source turbo spray and ESI interface. The liquid chromatography was a LC-20 AD series binary system equipped with an autosampler. The column used was C18 phenomenex (250×4.6 mm, 5 μm), flow rate 0.5 ml/min of mobile phase methanol: water (0.1% acetic acid), (10:90), wave length 254 nm and run time 25 min. The analytes were ionized by ESI in positive-ion mode (PI mode). Final ionization conditions were heated ionization temperature 435° C. Curtain gas Nitrogen 30 psi, particulate-free and CO₂-free air was used as ion source gas 1 at a flow rate 50 psi and ion source gas 2 at a flow rate 60 psi.

Sample Preparation for Ocimum sanctum Leaves Co₂ Extract:

Weigh about 50 mg of sample in a 50 ml volumetric flask, and dissolve by sonication in methanol (HPLC grade). Make the volume up to the mark with methanol filter through 0.2 um syringe filter.

Sample Preparation for Ocimum sanctum Leaves Water Extract:

Weigh about 500 mg of finely powdered sample in a 50 ml volumetric flask, and dissolve by sonication in water (HPLC grade). Make the volume up to the mark with water filter through 0.2 um syringe filter.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of this invention.

Abbreviated terms for following.
1) LOD Loss on drying
2) BD Bulk density
3) RMG Rapid Mixer Granulator
4) FBD Fluid Bed Drier
5) TVAC Total Viable Aerobic Count
6) NMT Not More Than
7) DT Disintegration Time

We claim:

1. A herbal solid formulation comprising a blend of Super Critical Fluid (CO2) extract, water extract and powder of herb Andrographis paniculata, Terminalia arjuna, Azadirachta indica, Trikatu (Zingiber officinalis, Piper longum, Piper nigrum), Tinospora cordifolia or Ocimum sanctum, wherein said blend of extract and said powder of herb is mixed in a ratio of about 1:0.5 to about 1:90.

2. The herbal solid formulation of claim 1, wherein said herbal solid formulation is essentially free of additives/excipients.

3. The process of claim 1, wherein the extract/powder of the herbs is obtained using bark & leaf of Terminalia arjuna, leaf & stem of Azadirachta indica rhizome, seed or pepper stem of Trikatu (Zingiber officinalis, Piper longum, Piper nigrum), leaf of Andrographis paniculata, stem of Tinospora cordifolia or leaf of Ocimum sanctum.

4. The herbal solid formulation of claim 1, wherein said solid formulation is preferably granules, tablet, caplet or capsule.

5. The herbal solid formulation of claim 1, wherein said solid formulation is a tablet.

6. The herbal solid formulation of claim 5, wherein the tablet is having hardness of about 2 to about 8 kg/cm2, a friability of less than about 1% and disintegration time is less than about 60 min.

7. The herbal solid formulation of claim 6, wherein the tablet is having disintegration time is less than about 30 min.

8. The herbal solid formulation of claim 1, wherein said solid formulation is a caplet.

9. The herbal solid formulation of claim 8, wherein the caplet comprising Andrographis paniculata containing 9 mg of Andrographolide or Azadirachta indica containing 18 mg of bitters including Nimbidin or Terminalia arjuna containing 0.25 mg of Arjunolic acid and 125 mg of total tannin or Ocimum sanctum containing 3.5 mg of total oleonolic acids or Trikatu containing 3 mg of Gingerols and 1.09 mg of Piperine or Tinospora cordifolia containing 24.6 mg of bitters including tinosporaside.

10. A process for preparing a herbal solid formulation as claimed in claim 1, comprising:
autoclaving powder of a herb;
granulating the autoclaved powder with a water extract of the herb;
lubricating the granulated mixture by adding the autoclaved powder of the herb; and
preparing the solid formulation.

11. The process of claim 10, wherein the extract of herb is obtained by employing Super Critical Fluid (CO2) extraction, percolation, hot soxhalation or refluxing.

12. The process of claim 11, wherein the percolation, hot soxhalation or refluxing method is performed in the presence of a solvent selected from water, grain alcohol or combinations thereof.

13. The process of claim 10, wherein the powder of herb is obtained by pulversing the herb to a powder having mesh size between about 20 to about 100.

14. The process of claim 10, wherein the extract of herb is obtained by pulversing the herb to a powder having mesh size between about 20 to 80.

15. The process of claim 10, wherein the granules are passed through a mesh having size between about 12 to 24.

16. The process of claim 10, wherein the granulation is carried out by employing a solvent system selected from water, grain alcohol or combinations thereof.
